(19) United States
(12) Patent Application Publication
(10) Pub. No.: US 2011/0297147 A1
(43) Pub. Date: Dec. 8, 2011

(54) SYSTEMS AND METHODS FOR CARDIAC ARREST PROTOCOL COMPLIANCE

(75) Inventors: Charles Lick, Roseville, MN (US); Keith Lurie, Roseville, MN (US); Tony Olson, Roseville, MN (US); Paul Satterlee, Roseville, MN (US)

(73) Assignee: ResQCor, Roseville, MN (US)

(21) Appl. No.: 12/792,333

(22) Filed: Jun. 2, 2010

(51) Int. Cl. A61M 16/00; A61B 19/02; B23P 17/04; A61M 5/00; G09B 23/28

(52) U.S. Cl. 128/202.16; 434/265; 29/428; 206/370

(57) ABSTRACT

A method for treating a person experiencing a cardiac arrest is disclosed. The method may include providing a kit having four sections and a visual medium. The first section may include a ventilation system. The second section may include a drug delivery system. The third section may include one set of drugs. The fourth section may include another set of drugs. The visual medium may include a two sets of instructions. The method may also include accessing the first section of the kit to ventilate the person and the second section of the kit to provide for drug delivery to the person. The method may include determining whether the person is experiencing a first or second cardiac arrest condition. The method may additionally include accessing the third or fourth section of the kit, based upon the determination, to provide one of the sets of drugs to the person.

### BACKGROUND OF THE INVENTION

This invention relates generally to the medical field. More specifically, the invention relates to the immediate treatment of individuals experiencing a cardiac arrest.

Cardiac arrest, which is the failure of the heart to continue circulating blood throughout the body, is the cause of about 15% of all deaths in Western countries. If the cardiac arrest occurs outside of a hospital environment, the survival rate of persons experiencing cardiac arrest is usually between 5 to 10%. Even when the cardiac arrests occur in a hospital environment, the survival rate only increases to about 15%. Because those who survive a cardiac arrest often survive for many years following the event, increasing survival rates of cardiac arrests is an important objective in the medical community.

Studies have found that during a stressful life-or-death cardiac arrest event even well trained emergency medical personnel will often fail to follow proven life-saving procedures in the heat of the moment. When emergency medical personnel follow proper cardiac arrest protocol, survival rates may increase by 250 to 500%.

Embodiments of the invention provide solutions to these and other problems related to cardiac arrest protocol compliance.

### BRIEF DESCRIPTION OF THE INVENTION

In one embodiment, a method for treating a person experiencing a cardiac arrest is provided. The method may include providing a kit. The kit may include a first section, a second section, a third section, a fourth section, and a visual medium. The first section may include a ventilation system. The second section may include a drug delivery system. The third section may include a first set of drugs. The fourth section may include a second set of drugs. The visual medium may include a first set of instructions and a second set of instructions. The method may also include accessing the first section of the kit to ventilate the person. The method may further include accessing the second section of the kit to provide for drug delivery to the person. The method may additionally include determining whether the person is experiencing a first cardiac arrest condition or a second cardiac arrest condition. The method may moreover include accessing, selectively, the third section of the kit or the fourth section of the kit, based at least in part upon the determination of whether the person is experiencing the first cardiac arrest condition or the second cardiac arrest condition, to provide either the first set of drugs or the second set of drugs to the person.

In another embodiment, a kit for treating a person experiencing a cardiac arrest is provided. The kit may include a first section including a ventilation system. The kit may also include a second section including a drug delivery system. The kit may further include a third section including a first set of drugs. The kit may additionally include a fourth section including a second set of drugs. The visual medium may at least partially separate the third section from the fourth section. The visual medium may also include a first set of instructions which has directions to be followed by at least one user for administering the first set of drugs. The visual medium may furthermore include a second set of instructions which has directions to be followed by the at least one user for administering the second set of drugs.

In another embodiment, a method for organizing a container for assisting in the treatment of a person experiencing a cardiac arrest is provided. The method may include providing a container, where the container has a first section, a second section, a third section, and a fourth section. The method may also include disposing a ventilation system into the first section. The method may further include disposing a drug delivery system into the second section. The method may additionally include disposing a first set of drugs into the third section. The method may moreover include disposing a second set of drugs into the fourth section. The method may also include coupling a movable visual medium having a first set of instructions and a second set of instructions between the third section and the fourth section. The movable visual medium may be coupled such that in a first position the visual medium hides the second set of instructions and at least a portion of the fourth section, and in a second position the visual medium hides the first set of instructions and at least a portion of the third section.

### BRIEF DESCRIPTION OF THE DRAWINGS

The present invention is described in conjunction with the appended figures:

FIG. 1 is an axonometric view of an embodiment of a kit of the invention for treating a person experiencing a cardiac arrest;

FIG. 2 is another axonometric view of the kit embodiment shown in FIG. 1 lying on its side in preparation for use on a person experiencing a cardiac arrest;

FIG. 3 is an axonometric view of the embodiment shown in FIG. 2, where the kit has been opened to expose the first and second sections of the kit;

FIG. 4 is an axonometric view of the embodiment shown in FIG. 3, where the kit has been further opened to expose the third section of the kit and a movable visual medium;

FIG. 5 is an axonometric view of the embodiment shown in FIG. 4, where the movable visual medium is being rotated to reveal the fourth section of the kit;

FIG. 6 is an axonometric view of the embodiment shown in FIG. 5, where the movable visual medium has been fully rotated to expose the fourth section of the kit;

FIG. 7A is an example set of instructions for performing treatment of a person experiencing a cardiac arrest and having an asystole condition and/or a pulseless electrical activity condition;

FIG. 7B is an example set of instructions for performing treatment of a person experiencing a cardiac arrest and having a ventricular fibrillation condition and/or a ventricular tachycardia condition;

FIG. 8 is a block diagram of a method of the invention for treating a person experiencing a cardiac arrest; and

FIG. 9 is a block diagram of a method of the invention for organizing a container for assisting in the treatment of a person experiencing a cardiac arrest.

In the appended figures, similar components and/or features may have the same numerical reference label. Further, various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix.

### DETAILED DESCRIPTION OF THE INVENTION

The ensuing description provides exemplary embodiments only, and is not intended to limit the scope, applicability or configuration of the disclosure. Rather, the ensuing description of the exemplary embodiments will provide those skilled in the art with an enabling description for implementing one or more exemplary embodiments. It will be well understood that various changes may be made in the function and arrangement of elements without departing from the spirit and scope of the invention as set forth in the appended claims.

Specific details are given in the following description to provide a thorough understanding of the embodiments. However, it will be understood by one of ordinary skill in the art that the embodiments may be practiced with or without these specific details. For example, systems, equipment, apparatuses, methods, processes, and other elements in the invention may be shown as components in block diagram form in order not to obscure the embodiments in unnecessary detail. In other instances, well-known processes, techniques, and devices may be shown without unnecessary detail in order to avoid obscuring the embodiments.

Also, it should be noted that individual embodiments may be described as a process which is depicted as a flowchart, a flow diagram, or a block diagram. Although any chart or diagram may describe the operations as a sequential process, many of the operations can be performed in parallel or concurrently. In addition, the order of the operations may be re-arranged. A procedure may be terminated when its operations are completed, but could have additional steps not discussed, or included in a figure. Furthermore, not all steps in any particularly described process may occur in all embodiments. A process may correspond to a method, a function, a procedure, etc.

Furthermore, embodiments of the invention may be implemented, at least in part, either manually or automatically. Manual or automatic implementations may be executed, or at least assisted, through the use of machines, hardware, software, firmware, middleware, microcode, hardware description languages, or any combination thereof.

In one embodiment of the invention, a method for treating a person experiencing a cardiac arrest is provided. The method may include providing a kit. The kit may include a first section, a second section, a third section, a fourth section, and a visual medium.

The first section may include a ventilation system. The ventilation system may include, merely by way of example, a ventilation mask, one or more oropharyngeal airways, and/or an impedance threshold system. In some embodiments, the first section may include an identifying label, possibly identifying the section as “Section 1” and/or an “Airway” section.

Other devices which may be included in the ventilation system, or other systems of the invention, include, merely by way of example, devices disclosed by: U.S. Pat. No. 7,210,480, issued on May 1, 2007, and entitled “SHOCK TREATMENT SYSTEMS AND METHODS;” U.S. patent application Ser. No. 11/690,065, filed on Mar. 22, 2007, and entitled “SYSTEMS AND METHODS FOR MODULATING AUTONOMIC FUNCTION” (published as U.S. Patent Application Pub. No. 2007/0255482); U.S. patent application Ser. No. 11/862,099, filed on Sep. 26, 2007, and entitled “BAG-VALVE RESUSCITATION FOR TREATING OF HYPOTENSION, HEAD TRAUMA, AND CARDIAC ARREST” (published as U.S. Patent Application Pub. No. 2008/0047555); U.S. patent application Ser. No. 11/735,924, filed on Apr. 16, 2007, and entitled “INTRATHORACIC PRESSURE LIMITER AND CPR DEVICE FOR REDUCING INTRACRANIAL PRESSURE AND METHODS OF USE” (published as U.S. Patent Application Pub. No. 2008/0255482); U.S. patent application Ser. No. 11/871,879, filed on Oct. 12, 2007, and entitled “VOLUME EXCHANGER VALVE SYSTEM AND METHOD TO INCREASE CIRCULATION DURING CPR” (published as U.S. Patent Application No. 2008/0257344); and U.S. patent application Ser. No. 12/141,864, filed on Jun. 18, 2008, and entitled “METHOD AND SYSTEM TO DECREASE INTRACRANIAL PRESSURE, ENHANCE CIRCULATION, AND ENCOURAGE SPONTANEOUS RESPIRATION” (published as U.S. Patent Application No. 2009/0020128). The entire disclosures of all of the above patents and patent applications are hereby incorporated by reference, for all purposes, as if fully set forth herein. Methods discussed in the above referenced patents and patent applications may also be incorporated by various methods of the instant invention.

The second section may include a drug delivery system. The drug delivery system may include, merely by way of example, an intraosseous (“IO”) infusion system, a saline container, and/or an intravenous (“IV”) delivery system. In some embodiments, the second section may include an identifying label, possibly identifying the section as “Section 2” and/or an “IV/IO” section.

The first section and/or second section may also include a timer, a code summary report, writing implements to complete the code summary report, a defibrillator, and an automatic chest compression device (for example LUCAS™ CPR, or LUCAS™ 2).

The third section may include a first set of drugs. The first set of drugs may include, merely by way of example, an epinephrine container (possibly in 1 milligram (mg) pre-fill quantity), an atropine container (possibly in 1 mg pre-fill quantity), and/or a vasopressin container (possibly two 20 unit vials, and also including a 6 cubic centimeter (cc) syringe with 18 gauge needle). In some embodiments, the third section may include an identifying label, possibly identifying the section as “Section 3” and/or an “Asystole/PEA” section.

In some embodiments, the first set of drugs may be organized and presented in a specified order in the third section, possibly in order of probable use during a cardiac arrest event as potentially described by one or more of the instruction sets present on the visual medium. Merely by way of example, the above described drugs, possibly in the configurations and quantities described, may be organized and presented in the following order: epinephrine, atropine, vasopressin, atropine, and atropine.

The fourth section may include a second set of drugs. The second set of drugs may include, merely by way of example, an epinephrine container (possibly in 1 mg pre-fill quantity), an amiodarone container (possibly in 150 mg vials, and also including a 6 cc syringe with 18 gauge needle), and a vasopressin container (possibly two 20 unit vials, and also including a 6 cubic centimeter (cc) syringe with 18 gauge needle). In some embodiments, the fourth section may include an identifying label, possibly identifying the section as “Section 4” and/or an “VTACH/VFIB” section.

In some embodiments, the second set of drugs may be organized and presented in a specified order in the fourth section, possibly in order of probable use during a cardiac arrest event as potentially described by one or more of the instruction sets present on the visual medium. Merely by way of example, the above described drugs, possibly in the configurations and quantities described, may be organized and presented in the following order: epinephrine, amiodarone, vasopressin, amiodarone, and epinephrine.

The visual medium may include a first set of instructions and a second set of instructions. In some embodiments, the visual medium may be movably coupled with the kit, possibly between the third section and the fourth section. In these as other embodiments, the visual medium may, for example, be movable between at least a first position and a second position. In a first position the visual medium may hide the second set of instructions and at least a portion of the fourth section. In a second position the visual medium may hide the first set of instructions and at least a portion of the third section.

In some embodiments, the first set of instructions and the second set of instructions each include directions to be followed at particular or approximate times after an at least approximate start time of the cardiac arrest. In these embodiments, the method may also include at least one user following the first set of instructions or the second set of instructions at the particular times.

In some embodiments, the first set of instructions and the second set of instructions may each include directions to be followed by different users. In these embodiments, the method may also include a first user ventilating the person according to the first set of instructions or the second set of instructions, and a second user providing drug delivery to the person according to the first set of instructions or the second set of instructions. In some embodiments, a third user may perform chest compressions on the person and/or operate an automatic chest compression device, a fourth user may administer defibrillation to the person, and/or a fifth user may lead the resuscitation team, and facilitate use of, or directly use, the timer and/or code summary report. In some embodiments, some of these functions may be combined and performed by fewer users.

In some embodiments, the visual medium may act as a “security flap,” hiding and limiting access to drugs that are not necessary for the set of instructions being followed on the currently visible side of the visual medium. Merely by way of example, if one set of instructions is for treatment of a person experiencing an asystole or pulseless electrical activity condition, the security flap/visual medium may at least partially, if not totally, obscure and inhibit access to drugs in the fourth section of the kit, which are for ventricular tachycardia and/or ventricular fibrillation conditions. In an opposite example, if the other set of instructions on the opposites side of the visual medium is for treatment of a person experiencing a ventricular tachycardia or ventricular fibrillation condition, the security flap/visual medium may at least partially, if not totally, obscure and inhibit access to drugs in the third section of the kit, which are for asystole and/or pulseless electrical activity conditions. In this way, different arrays of drugs, each for treatment of different conditions can be isolated from each other, reducing the possibility of use of the wrong drugs at the wrong time and/or for the wrong condition.

Additionally, in some embodiments, the first and/or second set of drugs may be provided in a tray or other container, disposable or otherwise, so that each set can be replaced in whole with a new tray or other container. The kit may include compartment(s) which accept these trays or other containers, thereby simplifying the process of refilling the kit after use.

In some embodiments the kit may also include a timer configured to provide time from an at least approximate start time of the cardiac arrest. In there embodiments, the method may also include starting the timer at the at least approximate start time of the cardiac arrest.

The method may also include accessing the first section of the kit to ventilate the person. The method may further include accessing the second section of the kit to provide for drug delivery to the person. In some embodiments, upon opening the kit from a closed condition, the kit may be in a first physical orientation, where in the first physical orientation the first section and the second section are immediately accessible.

The method may additionally include determining whether the person is experiencing a first cardiac arrest condition or a second cardiac arrest condition. In some embodiments, the first cardiac arrest condition may include an asystole condition and/or a pulseless electrical activity condition. In these or other embodiments, the second cardiac arrest condition may include a ventricular fibrillation condition and/or a ventricular tachycardia condition. In some embodiments, the kit may be reconfigurable to a second physical orientation from the closed condition via a transition through the first physical orientation, and wherein in the second physical orientation at least one of the third section and the fourth section is immediately accessible. In these or other embodiments, movement of the visual medium may change which of the third section and fourth section is immediately accessible to a user of the kit.

The method may moreover include accessing, selectively, the third section of the kit or the fourth section of the kit. In some embodiments, which of the third or fourth sections is accessed may be based at least in part upon the determination of whether the person is experiencing the first cardiac arrest condition or the second cardiac arrest condition. In these or other embodiments, the method may include providing either the first set of drugs or the second set of drugs to the person based on which of the third or fourth sections is accessed.

In some embodiments, when the kit is in the second physical orientation, the visual medium may selectively inhibit, at least partially, access to either the third section or the fourth section. In these or other embodiments, the visual medium may be movably coupled with the kit. When in a first position, the visual medium may hide the second set of instruction and at least a portion of the fourth section. When in a second position the visual medium may hide the first set of instructions and at least a portion of the third section.

In some embodiments the kit may also include at least one active compression decompression cardiopulmonary resuscitation assistance device. These devices may be any device known in the art, presently or in the future, for assisting in the delivery of active compression decompression cardiopulmonary resuscitation to a person. These device may be stored in any section of the kit, and in some embodiments will be stored in the first or second section of the kit. In these or other embodiments, the method may further include administering active compression decompression cardiopulmonary resuscitation using the active compression decompression cardiopulmonary resuscitation assistance device or devices in the kit.

Any particular kit may be customized for a given care provider, possibly due to their needs, preferences, and/or level of training of their staff. In these embodiments, the visual medium, and/or other guides, will provide instructions for the proper sequence of use of the contents of the customized kit. The organization and structure of the kit will also facilitate such use sequence in each of these customized kits.

In another embodiment of the invention, a kit for treating a person experiencing a cardiac arrest is provided. The kit may include a first section including a ventilation system. The kit may also include a second section including a drug delivery system. The kit may further include a third section including a first set of drugs. The kit may additionally include a fourth section including a second set of drugs. The visual medium may at least partially separate the third section from the fourth section. The visual medium may include a first set of instructions which has directions to be followed by at least one user for administering the first set of drugs. The visual medium may also include a second set of instructions which has directions to be followed by the at least one user for administering the second set of drugs. The kit may also include any of the elements or features described herein, supra or infra.

In another embodiment of the invention, a method for organizing a container for assisting in the treatment of a person experiencing a cardiac arrest is provided. The method may include providing a container, where the container has a first section, a second section, a third section, and a fourth section. The method may also include disposing a ventilation system into the first section. The method may further include disposing a drug delivery system into the second section. The method may additionally include disposing a first set of drugs into the third section. The method may moreover include disposing a second set of drugs into the fourth section. The method may also include coupling a movable visual medium having a first set of instructions and a second set of instructions between the third section and the fourth section. The movable visual medium may be coupled such that in a first position the visual medium hides the second set of instructions and at least a portion of the fourth section, an in a second position the visual medium hides the first set of instructions and at least a portion of the third section. The container may also include any of the elements or features described herein, supra or infra. Methods of organizing the container may further include any of the steps described herein, supra or infra.

Embodiments of the invention drive compliance with proven life saving protocols via a systems-based approach. This overall systems-based approach of providing the necessary apparatuses and drugs at the proper stages and times of treating a person experiencing cardiac arrest increases the likelihood that proven life saving protocols will be implemented in the proper sequence and at the proper times. Providing key instructions to the personal implementing such methods, along with co-sequenced and controlled access to the different apparatuses and drugs described by the instructions increases the likelihood that the instructions, and thus the proper protocols will be followed, thereby increasing the likelihood of an improved outcome for the person experiencing the cardiac arrest. Additionally, at a basic level, the total inclusion in the kit of all the identified components improves the immediate availability of the necessary components to personnel involved in the treatment of such persons over non-centralized supply of such components.

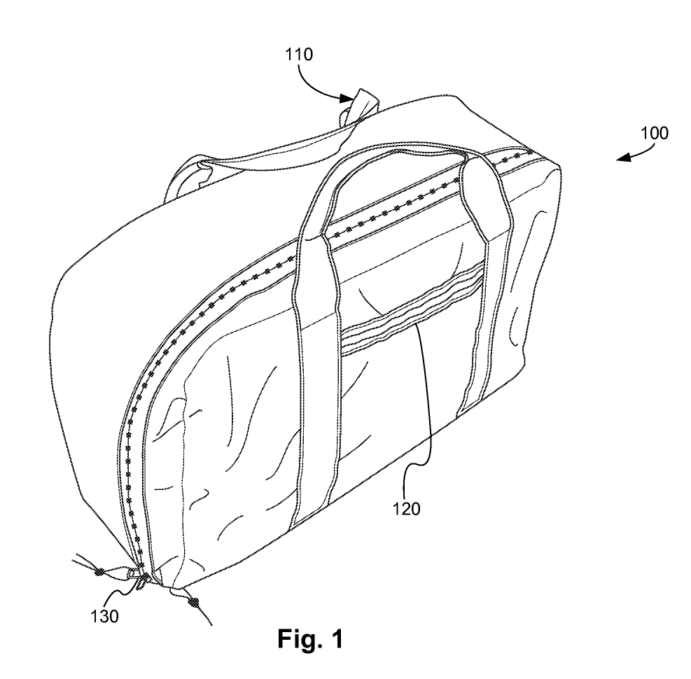

Turning now to FIG. 1, an axonometric view of an embodiment of a kit 100 of the invention for treating a person experiencing a cardiac arrest is shown. In this example, the exterior of kit 100 may include handles 110, as well as exterior pockets 120 to hold additional items as employed by users of kit 100. A zipper 130 may allow for access to the interior of kit 100, though other types of fastening/access methods may be employed, for example, Velcro™ and push button closures. In some embodiments, kit 100 may be constructed of natural or synthetic materials which are resistant to abrasive damage and fouling and/or may be easily cleaned. For example, synthetic fibers such as polymers (i.e. polyester, aramid, nylon, Ingeo™, etc.) or natural fibers such as wool, cotton, hemp which have been treated to increase durability may be employed. While in such embodiments kit 100 may be soft-sided, in these or other embodiments kit 100 may also be hard-sided through the use of composite, metal, or polymer materials.

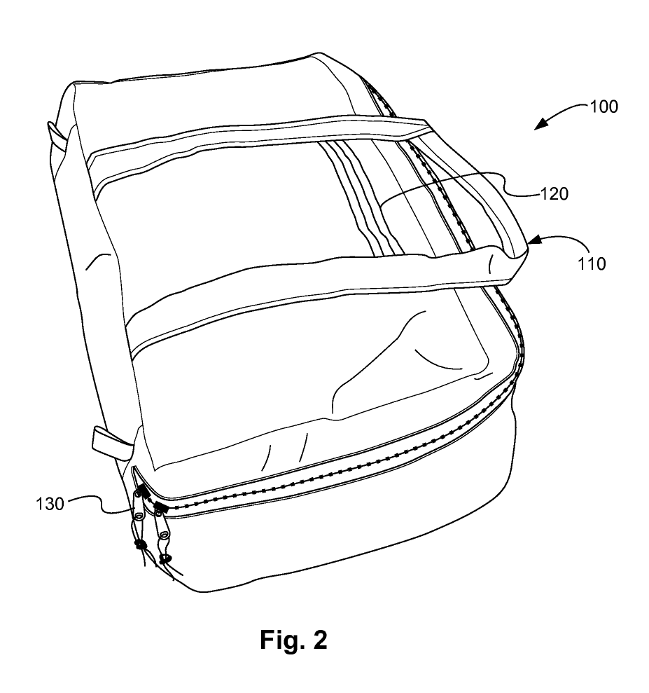

FIG. 2 shows another axonometric view of kit 100 shown in FIG. 1, but lying on its side in preparation for use on a person experiencing a cardiac arrest. In this position the kit is ready to be opened to be used during a cardiac arrest emergency.

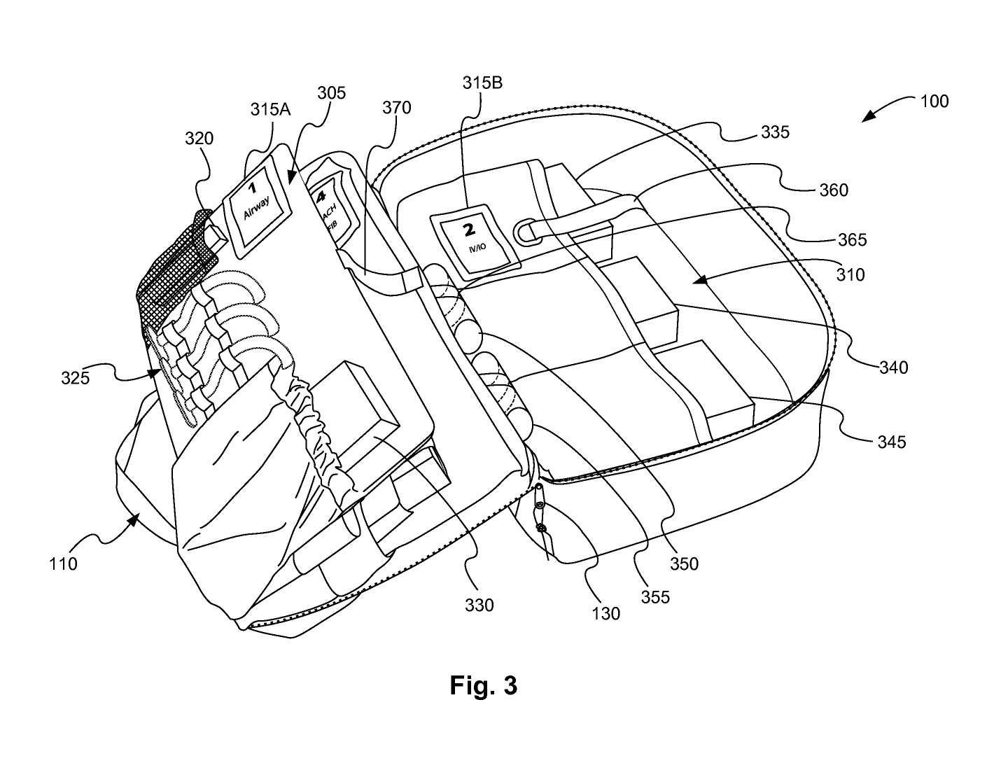

FIG. 3 shows an axonometric view of kit 100 shown from FIG. 2, where kit 100 has been opened to expose a first section 305 and a second section 310 of kit 100.

First section 305 may include a label 315A identifying it as first section 305 and as the “airway” section. This may assist a user in quickly identifying the purpose of first section 305 during a stressful cardiac arrest emergency. First section 305 may include an impedance threshold system 320 (for example, ResQPODT™), one or more oropharyngeal airways 325, and/or a ventilation mask 330.

Second section 310 may include a label 315B identifying it as second section 310 and as the “IV/IO” section. This may assist a user in quickly identifying the purpose of second section 310 during a stressful cardiac arrest emergency.

Second section 310 may include an intraosseous (“IO”) infusion system 335 (for example, EZIO™), an intravenous (“IV”) delivery system 340, and/or a saline bag 345. Second section 310 may also include a calcium chloride container 350 and a sodium bicarbonate container 355. Partially or fully detachable straps 360 and/or flexible straps 365 may secure contents within second section 310 or any other section.

First section 305 and/or second section 310 may also include a timer, a code summary report, writing implements to complete the code summary report, a defibrillator, and an automatic chest compression device (for example LUCAS™ CPR, or LUCAS™ 2).

A connecting device 370 may secure first section 305 to the kit until access to sections other than first section 305 and second section 310 is desired.

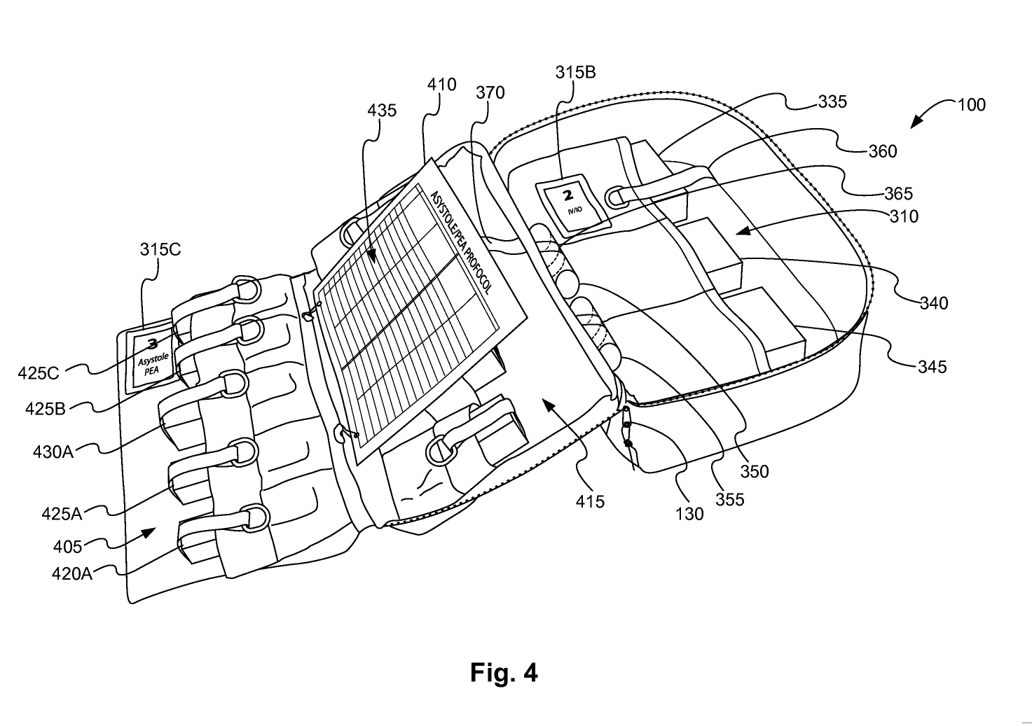

FIG. 4 shows an axonometric view of kit 100 shown in FIG. 3, where kit 100 has been further opened to expose a third section 405 of kit 100 and a movable visual medium 410. Visual medium 410 may at least partially, if not fully, obscure a fourth section 415 of kit 100.

Third section 405 may include a label 315C identifying it as third section 405 and as the “Asystole/PEA” section. This may assist a user in quickly identifying the purpose of third section 405 during a stressful cardiac arrest emergency.

Third section 405 of kit 100 may include a plurality of drugs. In this embodiment, the plurality of drugs may include epinephrine 420 (possibly in 1 mg pre-fill quantity), atropine 425 (possibly in 1 mg pre-fill quantity), and vasopressin 430 (possibly two 20 unit vials, and also including a 6 cubic centimeter (cc) syringe with 18 gauge needle).

A first set of instructions 435 may be included on one side of visual medium 410. First set of instructions 435 may provide instructions for how users of first section 305, second section 310 and third section 405 should employ the contents of those sections during a cardiac arrest emergency. First set of instructions may, in many embodiments, not provide instructions for use of fourth section 415, as that section may not be employed when section three 405 is utilized.

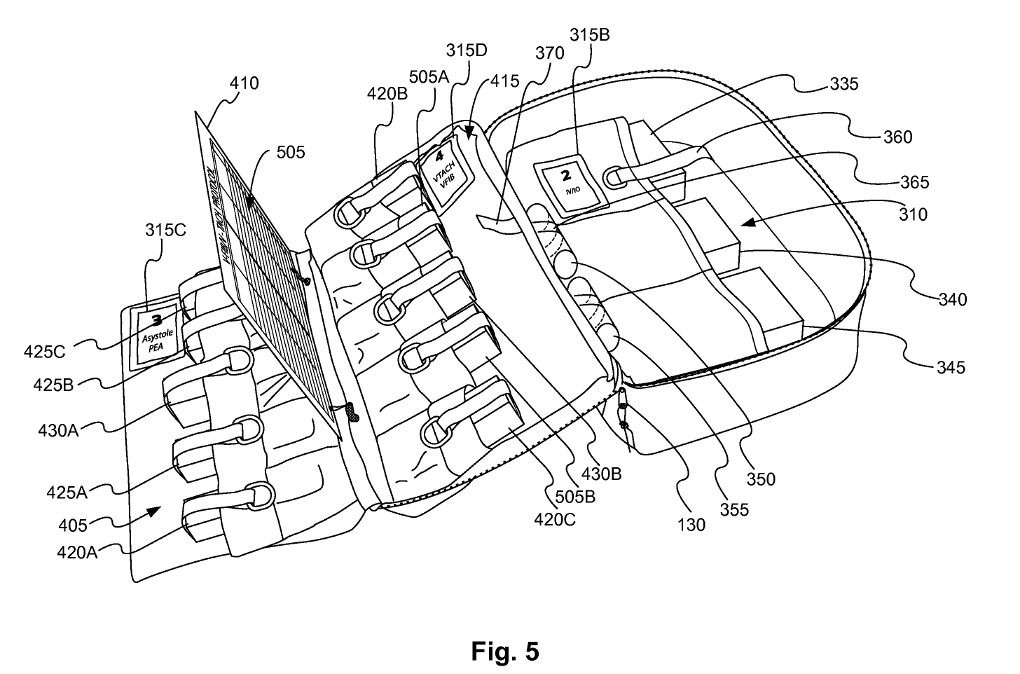

FIG. 5 shows an axonometric view of kit 100 shown in FIG. 4, where movable visual medium 410 is being rotated to reveal fourth section 415. A second set of instructions 505 is viewable on the other side of movable visual medium 410.

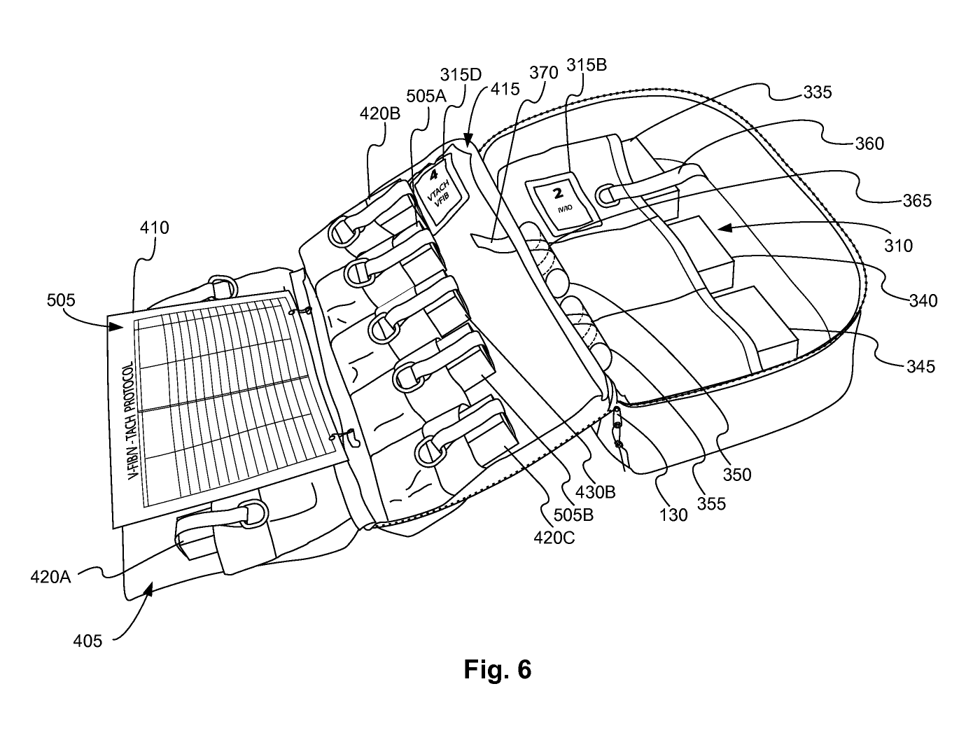

FIG. 6 shows an axonometric view of the kit 100 shown in FIG. 5, where movable visual medium 410 has been fully rotated to reveal fourth section 415. Visual medium 410 may at least partially, if not fully, obscure third section 405 of kit 100.

Fourth section 415 may include a label 315D identifying it as fourth section 415 and as the “VTACH/VFIB” section. This may assist a user in quickly identifying the purpose of fourth section 415 during a stressful cardiac arrest emergency.

Fourth section 415 of kit 100 may include a plurality of drugs. In this embodiment, the plurality of drugs may include epinephrine 420 (possibly in 1 mg pre-fill quantity), amiodarone 505 (possibly in 150 mg vials, and also including a 6 cc syringe with 18 gauge needle), and vasopressin 430 (possibly two 20 unit vials, and also including a 6 cubic centimeter (cc) syringe with 18 gauge needle).

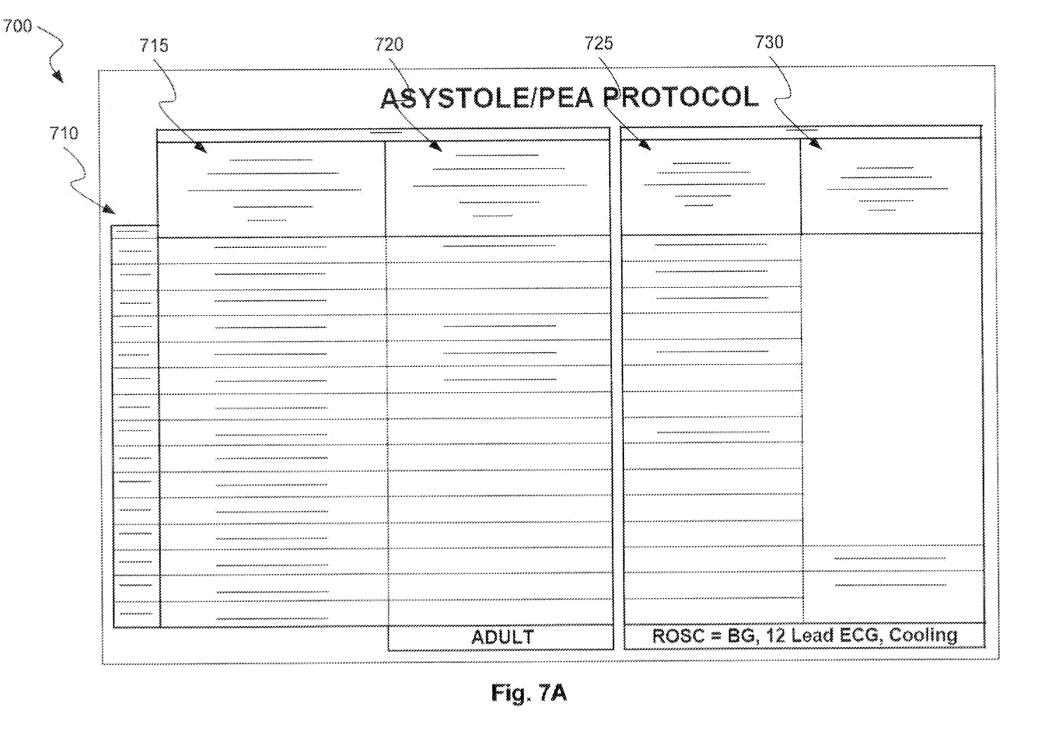

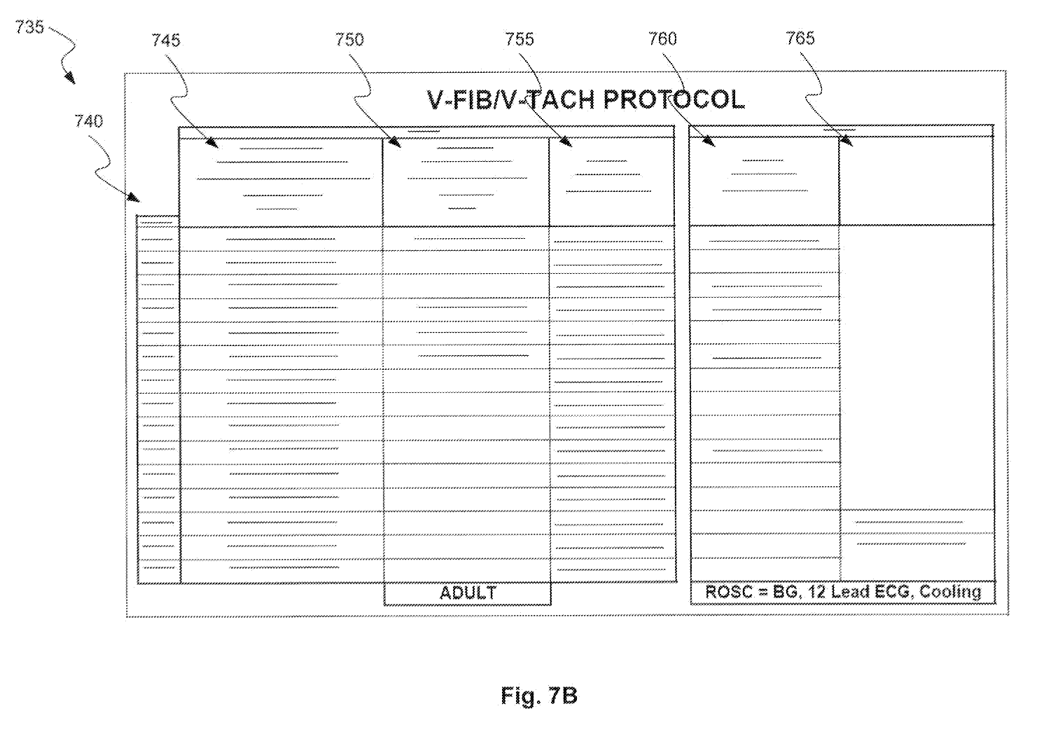

FIG. 7A shows a first example set of instructions 700 for performing treatment of a person experiencing a cardiac arrest and having an asystole condition and/or a pulseless electrical activity condition. First example set of instructions 700 may include a listing of times 710 after a cardiac arrest for which different functions should possibly be performed. These functions could be described by cardiopulmonary resuscitation sub-instructions 715, airway sub-instructions 720 which might implement articles stored in first section 305 of kit 100, drug administering sub-instructions 725 which might implement articles stored in second section 310 and third section 405 of kit 100, and resuscitation leader sub-instructions 730.

FIG. 7B shows a second example set of instructions 735 for performing treatment of a person experiencing a cardiac arrest and having a ventricular fibrillation condition and/or a ventricular tachycardia condition. Second example set of instructions 735 may include a listing of times 740 after a cardiac arrest for which different functions should possibly be performed. These functions could be described by cardiopulmonary resuscitation sub-instructions 745, airway sub-instructions 750 which might implement articles stored in first section 305 of kit 100, defibrillation sub-instructions 755, drug administering sub-instructions 760 which might implement articles stored in second section 310 and third section 405 of kit 100, and resuscitation leader sub-instructions 765.

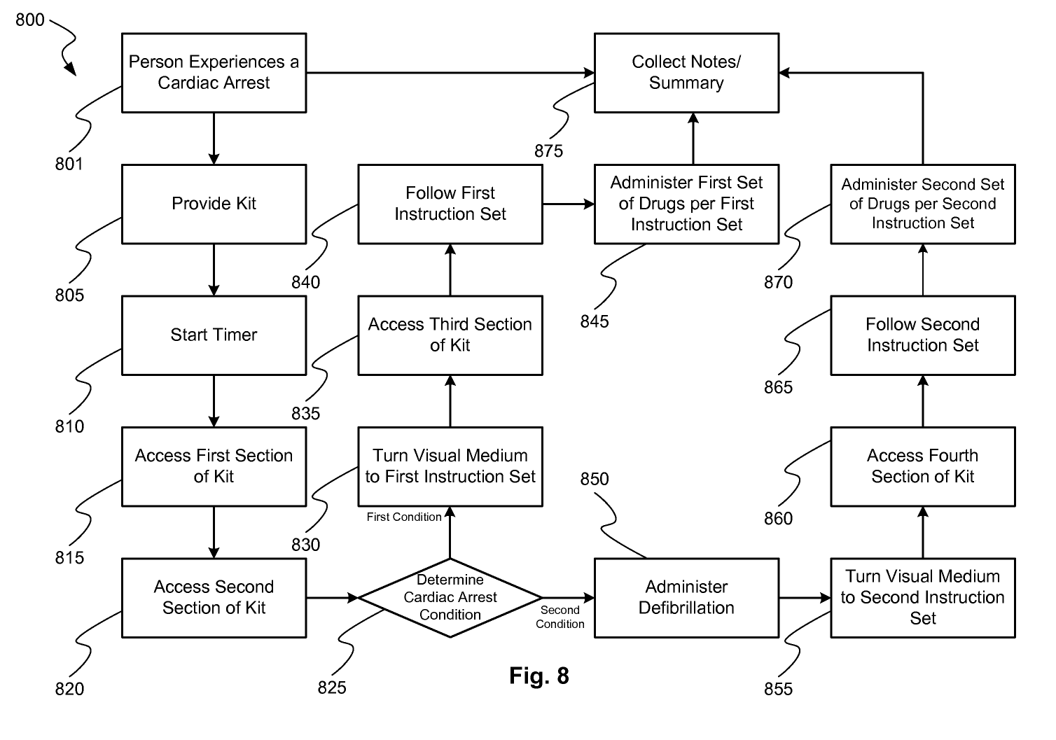

FIG. 8 shows a block diagram of a method 800 of the invention for treating a person experiencing a cardiac arrest. At block 801, the cardiac arrest occurs. At block 805, a kit 100 is provided. Kit 100 may be substantially as described above and herein, and may have a first section 305, a second section 310, a third section 405, and a third section 415. First section 305 may include a ventilation system. Second section 310 may include a drug delivery system. Third section 405 may include a first set of drugs. Fourth section 415 may include a second set of drugs. Kit 100 may also include a timer, possibly located in first section 305 or second section 310 so it is immediately accessible upon opening of kit 100.

At block 810, the timer from kit 100 is retrieved and started as soon as possible after the cardiac arrest. If it has been a known time since the cardiac arrest occurred, the timer may be adjusted accordingly to reflect the true time elapsed since the cardiac arrest occurred. At block 815, method 800 may include accessing first section 305 of the kit to ventilate the person. At block 820, method 800 may include accessing second section 315 of kit 100 to provide for drug delivery to the person.

At block 825, method 800 may include determining whether the person is experiencing a first cardiac arrest condition or a second cardiac arrest condition. The first cardiac arrest condition, merely by way of example, may be an asystole condition and/or a pulseless electrical activity condition. The second cardiac arrest condition, merely by way of example, may be a ventricular fibrillation condition and/or a ventricular tachycardia condition.

If it is determined at block 825 that a first cardiac arrest condition, possibly an asystole condition and/or a pulseless electrical activity condition, exists in the person experiencing the cardiac arrest, then at block 830 a visual medium 410 may be orientated such a first set of instructions 435 on visual medium 410 is visible, and a second set of instructions 505 on visual medium 410 is not. Orientation of visual medium 410 at block 830 may also cause fourth section 415 of kit 100 to be at least partially obscured.

Upon properly orientating visual medium 410, at block 835 third section 405 of kit 100 is accessed. At block 840, first set of instructions 435 is followed, and at block 845 a first set of drugs 420, 425, 430 is administered accordingly.

If it is determined at block 825 that a second cardiac arrest condition, possibly a ventricular fibrillation condition and/or a ventricular tachycardia condition, exists in the person experiencing the cardiac arrest, then at block 850 defibrillation may be administered, possibly with a portable defibrillator supplied in kit 100.

At block 855, visual medium 410 may be orientated such second set of instructions 505 on visual medium 410 is visible, and first set of instructions 435 on visual medium 410 is not. Orientation of visual medium 410 at block 855 may also cause third section 405 of kit 100 to be at least partially obscured.

Upon properly orientating visual medium 410, at block 860 fourth section 405 of kit 100 is accessed. At block 865, second set of instructions 505 is followed, and at block 870 a second set of drugs 420, 430, 505 is administered accordingly.

At block 875, a code summary report may be completed by one or more of the persons administering care to the person experiencing the cardiac arrest. This may occur during or after administration of care.

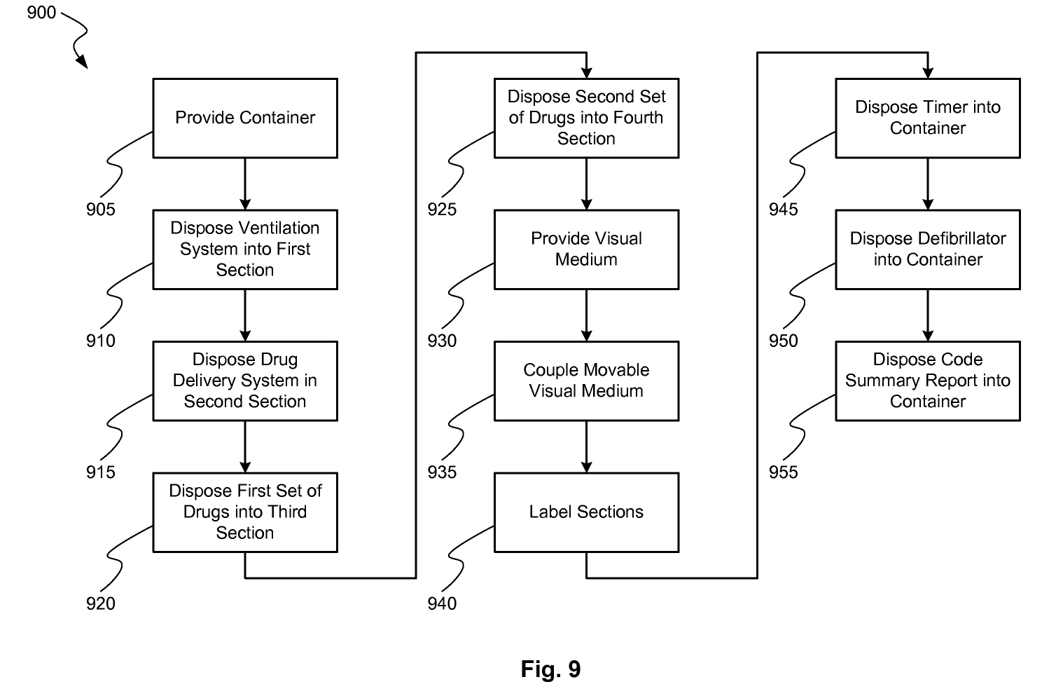

FIG. 9 shows a block diagram of a method 900 of the invention for organizing a container for assisting in the treatment of a person experiencing a cardiac arrest.

At block 905, a container may be provided. In some embodiments, the container may have multiple sections. In exemplary embodiments, the container may have four sections.

At block 910, a ventilation system may be disposed within the first section. At block 915, a drug delivery system may be disposed within the second section. In an alternative embodiment, the kit may have three sections and the ventilation system and drug delivery system may both be disposed within a first section of the kit.

At block 920, a first set of drugs may be disposed within another section of the kit, possibly the third section as described above. In embodiments with three sections, the first set of drugs may be disposed within the second section. At block 925, a second set of drugs may be disposed within another section of the kit, possibly the fourth section as described above. In embodiments with three section, the second set of drugs may be disposed within the third section.

At block 930, a visual medium may be provided. The visual medium may have alternate sets of instructions as discussed herein. At block 935, the visual medium may be coupled with the kit such that it can be moved and alternatively (1) reveal a first set of instructions and obscure the second set of drugs, or (2) reveal a second set of instructions and obscure the first set of drugs.

At block 940, the sections of the kit may be labeled so that medical personnel can quickly identify the likely contents of a section during a medical emergency. At block 945, a timer may be disposed within the container. In some embodiments, the timer may be disposed within the first or second sections of the kit so it may be quickly accessed immediately after the onset of a cardiac arrest.

At block 950, a defibrillator may be disposed within the container. In some embodiments, the defibrillator may be disposed within the first or second sections of the kit so it may be quickly accessed immediately after the onset of a cardiac arrest. At block 955, a code summary report worksheet may be disposed within the container. Writing implements such as pens and pencils may also be disposed within the container for use in completing the code summary report during or immediately after the cardiac arrest event. In some embodiments an audio and/or visual recording device may also be disposed within the kit for recording the response to a cardiac arrest.

The invention has now been described in detail for the purposes of clarity and understanding. However, it will be appreciated that certain changes and modifications may be practiced within the scope of the appended claims.

What is claimed is:

1. A method for treating a person experiencing a cardiac arrest, wherein the method comprises:
providing a kit, wherein the kit comprises:
a first section comprising a ventilation system;
a second section comprising a drug delivery system;
a third section comprising a first set of drugs;
a fourth section comprising a second set of drugs; and
a visual medium comprising:
a first set of instructions; and
a second set of instructions;
accessing the first section of the kit to ventilate the person;
accessing the second section of the kit to provide for drug delivery to the person;
determining whether the person is experiencing a first cardiac arrest condition or a second cardiac arrest condition; and
accessing, selectively, the third section of the kit or the fourth section of the kit, based at least in part upon the determination of whether the person is experiencing the first cardiac arrest condition or the second cardiac arrest condition, to provide either the first set of drugs or the second set of drugs to the person.

2. The method for treating a person experiencing a cardiac arrest of claim 1, wherein the visual medium is movably coupled with the kit, and wherein:
in a first position the visual medium hides the second set of instructions and at least a portion of the fourth section; and
in a second position the visual medium hides the first set of instructions and at least a portion of the third section.

3. The method for treating a person experiencing a cardiac arrest of claim 1, wherein the kit further comprises an identifying label on each of the first section, the second section, the third section, and the fourth section.

4. The method for treating a person experiencing a cardiac arrest of claim 1, wherein:
the kit further comprises a timer configured to provide time from an at least approximate start time of the cardiac arrest; and
the method further comprises starting the timer at the at least approximate start time of the cardiac arrest.

5. The method for treating a person experiencing a cardiac arrest of claim 1, wherein:
the first set of instructions and the second set of instructions each include directions to be followed at particular times after an at least approximate start time of the cardiac arrest; and
the method further comprises at least one user following the first set of instructions or the second set of instructions at the particular times.

6. The method for treating a person experiencing a cardiac arrest of claim 1, wherein:
the first set of instructions and the second set of instructions each include directions to be followed by different users; and
the method further comprises:
a first user ventilating the person according to the first set of instructions or the second set of instructions; and
a second user providing drug delivery to the person according to the first set of instructions or the second set of instructions.

7. The method for treating a person experiencing a cardiac arrest of claim 1, wherein the ventilation system comprises:
a ventilation mask;
an oropharyngeal airway; and
an impedance threshold system.

8. The method for treating a person experiencing a cardiac arrest of claim 1, wherein the drug delivery system comprises:
an intraosseous infusion system;
a saline container; and
an intravenous delivery system.

9. The method for treating a person experiencing a cardiac arrest of claim 1, wherein the first set of drugs comprises:
an epinephrine container;
an atropine container; and
a vasopressin container.

10. The method for treating a person experiencing a cardiac arrest of claim 1, wherein the second set of drugs comprises:
an epinephrine container;
an amiodarone container; and
a vasopressin container.

11. The method for treating a person experiencing a cardiac arrest of claim 1, wherein:
the first cardiac arrest condition comprises a selection from the group consisting of:
an asystole condition; and
a pulseless electrical activity condition; and
the second cardiac arrest condition comprises a selection from the group consisting of:
a ventricular fibrillation condition; and
a ventricular tachycardia condition.

12. The method for treating a person experiencing a cardiac arrest of claim 1, wherein the kit further comprises an active compression decompression cardiopulmonary resuscitation assistance device, and the method further comprises administering active compression decompression cardiopulmonary resuscitation using the active compression decompression cardiopulmonary resuscitation assistance device.

13. A kit for treating a person experiencing a cardiac arrest, wherein the kit comprises:
a first section comprising a ventilation system;
a second section comprising a drug delivery system;
a third section comprising a first set of drugs;
a fourth section comprising a second set of drugs; and
a visual medium, wherein the visual medium at least partially separates the third section from the fourth section, and comprises:
a first set of instructions comprising directions to be followed by at least one user for administering the first set of drugs; and
a second set of instructions comprising directions to be followed by the at least one user for administering the second set of drugs.

14. The kit for treating a person experiencing a cardiac arrest of claim 13, wherein upon opening the kit from a closed condition, the kit is in a first physical orientation, and wherein in the first physical orientation the first section and the second section are immediately accessible.

15. The kit for treating a person experiencing a cardiac arrest of claim 14, wherein the kit is reconfigurable to a second physical orientation from the closed condition via a transition through the first physical orientation, and wherein in the second physical orientation at least one of the third section and the fourth section is immediately accessible.

16. The kit for treating a person experiencing a cardiac arrest of claim 15, wherein in the second physical orientation the visual medium selectively inhibits, at least partially, access to either the third section or the fourth section.

17. The kit for treating a person experiencing a cardiac arrest of claim 13, wherein the visual medium is movably coupled with the kit, and wherein:
in a first position the visual medium hides the second set of instruction and at least a portion of the fourth section; and
in a second position the visual medium hides the first set of instructions and at least a portion of the third section.

18. The kit for treating a person experiencing a cardiac arrest of claim 13, wherein the kit further comprises an identifying label on each of the first section, the second section, the third section, and the fourth section.

19. The kit for treating a person experiencing a cardiac arrest of claim 13, wherein the kit further comprises a timer configured to provide time from an at least approximate start time of the cardiac arrest.

20. A method for organizing a container for assisting in the treatment of a person experiencing a cardiac arrest, wherein the method comprises:
providing a container, wherein the container comprises:
a first section;
a second section;
a third section; and
a fourth section;
disposing a ventilation system into the first section;
disposing a drug delivery system into the second section;
disposing a first set of drugs into the third section;
disposing a second set of drugs into the fourth section; and
coupling a movable visual medium having a first set of instructions and a second set of instructions between the third section and the fourth section such that:
in a first position the visual medium hides the second set of instructions and at least a portion of the fourth section; and
in a second position the visual medium hides the first set of instructions and at least a portion of the third section.

21. The method for organizing a container for assisting in the treatment of a person experiencing a cardiac arrest of claim 20, wherein the method further comprises:
labeling the first section;
labeling the second section;
labeling the third section; and
labeling the fourth section.
